(19) United States
(12) Patent Application Publication
(10) Pub. No.: US 2020/0060704 A1
(43) Pub. Date: Feb. 27, 2020

(54) DIRECT CONTACT SHOCKWAVE TRANSDUCER

(71) Applicant: Moshe Ein-Gal, Ramat Hasharon (IL)

(72) Inventor: Moshe Ein-Gal, Ramat Hasharon (IL)

(21) Appl. No.: 16/106,362

(22) Filed: Aug. 21, 2018

(51) Int. Cl. A61B 17/225; A61H 23/00

(52) U.S. Cl. CPC A61B 17/225; A61H 23/008

(57) ABSTRACT

A shockwave system includes a shockwave transducer that has a shockwave generating portion operable to generate shockwaves and a transducer interface. The transducer interface includes a container whose outer contour is configured to directly contact an inner wall of a cavity in a patient. The container is at least partially filled with a liquid capable of transmitting the shockwaves from the shockwave generating portion to the inner wall of the cavity. The container completely surrounds the shockwave generating portion

### FIELD OF THE INVENTION

The present invention relates to a method and a system for shockwave generation and shockwave treatment, and particularly a shockwave generation system with a transducer interface which includes a container that completely surrounds the shockwave generating portion.

### BACKGROUND OF THE INVENTION

Prior art electromagnetic, electrohydraulic and piezoelectric shockwaves transducers are configured to produce pressure waves in a propagation medium, typically water. Sufficient propagation distance enables focusing and shockwaves formation. The transducers and the focusing means are generally circularly symmetric and are configured to produce generally spherical converging waves.

U.S. Pat. No. 9,555,267 to Ein-Gal describes a system for producing, shaping and coupling pulses of pressure waves to a patient by direct contact of one or more transducers to a part of the patient's body. The pressure waves do not propagate through a propagating liquid. Instead, the transducer incorporates a solid interface as the propagating medium. Coupling the transducer to the patient at such a close proximity is implemented with electrically safe and bio-compatible contact as well as with mechanically efficient matching of respective acoustic impedances of the patient and the transducer. Waves emanating from a single transducer are not necessarily focused. Focusing is obtainable by various methods, such as cupping the membrane, by shaping the solid interface as a lens or by configuring multiple transducers as a phased array where the transducers are respectively energized according to a timed sequence so as to assure simultaneous arrival of the waves from the respective transducers at a desired focal region.

Throughout the disclosure, the terms shockwave, pressure pulse and pressure wave are used interchangeably.

### SUMMARY OF THE INVENTION

The present invention seeks to provide a novel shockwave treatment system and method, as is described more in detail hereinbelow, which has use in many medical applications, such as but not limited to, lithotripsy, orthopedics, treating pathological tissue conditions and many others, in particular, applications to soft tissue. The shockwave system has a transducer interface, which includes a container that completely surrounds the shockwave generating portion.

There is provided in accordance with a non-limiting embodiment of the invention a shockwave system including a shockwave transducer including a shockwave generating portion operable to generate shockwaves and a transducer interface, wherein the transducer interface includes a container whose outer contour is configured to directly contact an inner wall of a cavity in a patient, the container being at least partially filled with a liquid capable of transmitting the shockwaves from the shockwave generating portion to the inner wall of the cavity, and wherein the container completely surrounds the shockwave generating portion

In accordance with a non-limiting embodiment of the invention a heater heats the outer contour of the container. The heater may be located in a wall of the container or in the liquid.

In accordance with a non-limiting embodiment of the invention a thermal sensor is in communication with a controller for controlling temperature of the outer contour of the container.

In accordance with a non-limiting embodiment of the invention the liquid is in fluid communication with a reservoir and a controller for controlling volume of the liquid.

In accordance with a non-limiting embodiment of the invention a magnet is operable to induce a magnetic field in the shockwave generating portion.

In accordance with a non-limiting embodiment of the invention a pulser is operable to deliver electrical current pulses to the shockwave generating portion, wherein the shockwave generating portion generates shockwaves in response to the magnetic field and the electric current pulses.

In accordance with a non-limiting embodiment of the invention the shockwaves propagate radially in the liquid in the container.

In accordance with a non-limiting embodiment of the invention walls of the container are flexible and the container is inflatable.

The shockwave generating portion may be cylindrical or conical.

In accordance with a non-limiting embodiment of the invention the transducer interface has acoustic impedance not lower than that of the inner wall of the cavity and not higher than that of the shockwave generating portion.

In accordance with a non-limiting embodiment of the invention there is provided a method for applying pressure pulses to an inner wall of a cavity of a patient, including introducing the shockwave transducer into the cavity of the patient, and using the shockwave generating portion to generate shockwaves through the transducer interface to the inner wall of the cavity.

### BRIEF DESCRIPTION OF THE DRAWINGS

The present invention will be understood and appreciated more fully from the following detailed description taken in conjunction with the drawings in which:

FIG. 1 is a simplified illustration of a system for pressure wave generation or treatment, constructed and operative in accordance with an embodiment of the invention.

### DETAILED DESCRIPTION OF EMBODIMENTS

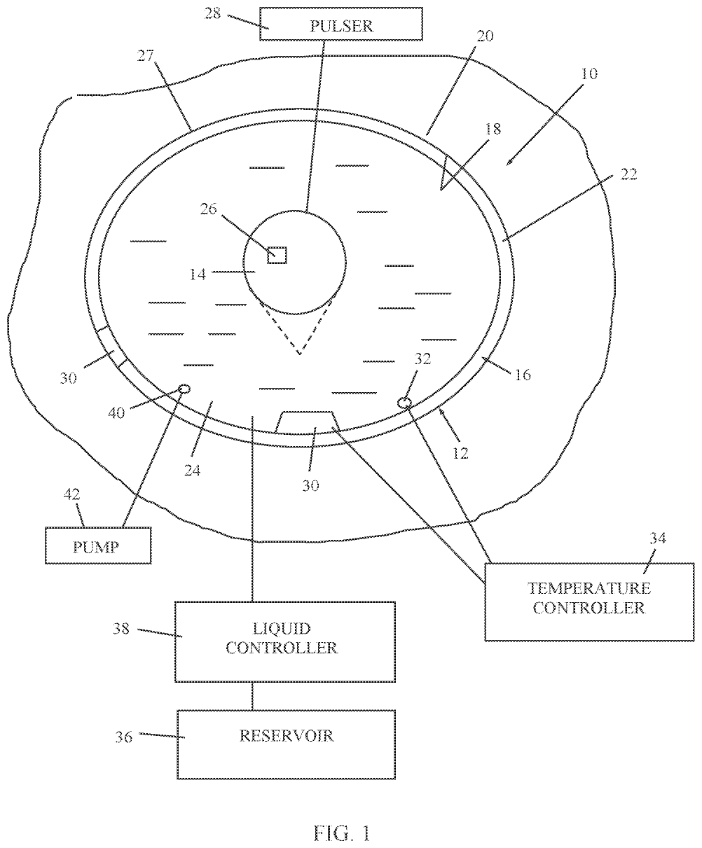

Reference is now made to FIG. 1, which illustrates a system 10 for shockwave generation or treatment, constructed and operative in accordance with a non-limiting embodiment of the invention.

System 10 includes a shockwave transducer 12 that includes a shockwave generating portion 14 and a transducer interface 16 arranged to contact an inner wall 18 of a cavity 20 of a patient.

The transducer interface 16 includes a container 22 whose outer contour is configured to directly contact the inner wall 18 of cavity 20. Container 22 is at least partially filled with a liquid 24 capable of transmitting the shockwaves from the shockwave generating portion 14 to the inner wall 18 of cavity 20. The container 22 completely surrounds the shockwave generating portion 14.

In accordance with a non-limiting embodiment of the invention walls of the container 22 are flexible and the container 22 is inflatable.

The shockwave generating portion 14 of shockwave transducer 12 may include, without limitation, an electrical-to-shockwave energy converter (e.g., electro-hydraulic, electromagnetic or piezoelectric) that generates shockwaves (acoustic pressure pulses). The shockwave generating portion 14 may be cylindrical or conical (shown in broken lines) or other shapes. The shockwaves may propagate radially in the liquid 24 in the container 22.

In one non-limiting example, shockwave generating portion 14 may be a membrane configured to communicate with a magnet 26 (may be an electromagnet), which induces a magnetic field in the membrane, parallel to the outer surface of the membrane. Additionally or alternatively, the shockwave generating portion 14 may communicate with a pulser (electric pulse generator) 28. Pulser 28 delivers pulses of electrical current. For example, the current orientation may be parallel to the outer surface of the membrane of the shockwave generating portion 14 and but not necessarily parallel to the magnetic field in the membrane. Shockwave generating portion 14 is configured to repel and deliver pressure pulses to the liquid 24 in response to the interaction between the magnetic field and the current pulses in the membrane. The membrane may be planar, concave, convex or other shapes.

In the example of the magnet 26 being an electromagnet, it may include an electromagnet induction coil and the pulser 28 deliver current pulses to the membrane by induction from a pulser induction coil.

Imaging apparatus (not shown) may be provided for imaging the delivery of the shockwaves to the tissue.

The outer contour (wall) of container 22 is preferably made of an electrically safe and bio-compatible material that exhibits mechanically efficient matching of respective acoustic impedances of the patient and the bio-compatible material. Preferably, the bio-compatible material of transducer interface 16 has an acoustic impedance not lower than that of the tissue of the cavity's inner wall and not higher than that of shockwave generating portion 14, and most preferably close (within 20%) to the geometric mean of the two.

The acoustic impedance (Z) of a material is defined as the product of its density (φ and acoustic velocity (V), that is, Z=ρ*V, and is measured in Rayls (kg/(sec·m²)] or more conveniently in MegaRayls (MRayls).

The outer contour (wall) of container 22 may include multiple cascaded layers, each incorporating a respective acoustic impedance so as to provide adequate waves propagation in the transducer interface 16. The layer contacting the patient incorporates acoustic impedance close to that of the inner wall tissue, so as to minimize waves' reflection at the interface with the patient and the associated damage to the tissue.

The outer wall of container 22 may be covered with a medication 27 to be transferred to the cavity tissue (e.g., for treating bladder cancer).

In one non-limiting embodiment of the invention, system 10 combines shockwave transducer 12 with one or more other transducers for delivering energy, such as but not limited to, an optical energy, an ultrasonic energy, an RF energy, a magnetic energy, a microwave energy generator and/or mechanical energy generator (e.g., a spring or oscillating mass).

In accordance with a non-limiting embodiment of the invention a heater 30 heats the outer contour of container 22. The heater 30 may be located in a wall of container 22 or in the liquid 24. Heater 30 may be, without limitation, a resistance heater, thermoelectric heater, induction heater and many more. The heater 30 may transfer heat, by convection through liquid 24 and then by conduction through the wall thickness of the container 22, to the cavity tissue.

A thermal sensor 32 (e.g., without limitation, a thermistor or thermocouple) may be in communication with a controller 34, which is coupled to heater 30, for sensing and controlling temperature of the outer contour of the container 22. In this manner, the tissue may be treated not only with shockwaves but also with thermal energy.

In accordance with a non-limiting embodiment of the invention the liquid 24 is in fluid communication with a reservoir 36 and a fluid controller 38 for controlling volume of the liquid 24. In this manner, the amount of liquid in the container can be monitored and increased or decreased in accordance with a treatment plan.

Additionally or alternatively, the liquid may be in communication with a pressure sensor 40 and a pump 42 for controlling the pressure on the cavity wall (e.g., for treating calcified arteries).

What is claimed is:

1. A shockwave system comprising:
a shockwave transducer comprising a shockwave generating portion operable to generate shockwaves and a transducer interface;
wherein said transducer interface comprises a container whose outer contour is configured to directly contact an inner wall of a cavity in a patient, said container being at least partially filled with a liquid capable of transmitting said shockwaves from said shockwave generating portion to the inner wall of the cavity; and
wherein said container completely surrounds said shockwave generating portion.

2. The shockwave system according to claim 1, further comprising a heater configured to heat the outer contour of said container.

3. The shockwave system according to claim 2, wherein said heater is located in a wall of said container.

4. The shockwave system according to claim 2, wherein said heater is located in said liquid.

5. The shockwave system according to claim 4, further comprising a thermal sensor in communication with a controller, which is coupled to said heater, for controlling temperature of the outer contour of said container.

6. The shockwave system according to claim 1, wherein said liquid is in fluid communication with a reservoir and a controller for controlling volume of said liquid.

7. The shockwave system according to claim 1, further comprising a magnet operable to induce a magnetic field in said shockwave generating portion.

8. The shockwave system according to claim 7, further comprising a pulser operable to deliver electrical current pulses to said shockwave generating portion, wherein said shockwave generating portion generates shockwaves in response to said magnetic field and said electric current pulses.

9. The shockwave system according to claim 1, wherein said shockwaves propagate radially in said liquid in said container.

10. The shockwave system according to claim 1, wherein walls of said container are flexible and said container is inflatable.

11. The shockwave system according to claim 1, wherein said shockwave generating portion is cylindrical.

12. The shockwave system according to claim 1, wherein said shockwave generating portion is conical.

13. The system according to claim 1, wherein said transducer interface has acoustic impedance not lower than that of the inner wall of the cavity and not higher than that of said shockwave generating portion.

14. The system according to claim 1, wherein said liquid is in communication with a pressure sensor and a pump.

15. A method for applying pressure pulses to an inner wall of a cavity of a patient, the method comprising:
introducing a shockwave transducer as in claim 1 into a cavity of a patient; and
using said shockwave generating portion to generate shockwaves through said transducer interface to an inner wall of the cavity.
